(19) United States
(12) Patent Application Publication
(10) Pub. No.: US 2009/0076201 A1
(43) Pub. Date: Mar. 19, 2009

(54) (METH) ACRYLOYLOXYTETRAHYDROFURANS AND PROCESS FOR PRODUCTION THEREOF

(75) Inventors: Haruhiko Kusaka, Kanagawa (JP); Hiroko Takahashi, Kanagawa (JP); Yuji Ohgomori, Kanagawa (JP); Kaoru Hamashima, Fukuoka (JP); Gan Wen, Fukuoka (JP); Taketoshi Naito, Kanagawa (JP)

Correspondence Address: OBLON, SPIVAK, MCCLELLAND MAIER & NEUSTADT, P.C., 1940 DUKE STREET, ALEXANDRIA, VA, 22314, US

(73) Assignee: Mitsubishi Chemical Corporation, Minato-ku, Tokyo (JP)

(21) Appl. No.: 11/915,043

(22) PCT Filed: Mar. 16, 2006
(86) PCT No.: PCT/JP2006/305294

§ 371 Date: May 9, 2008

(30) Foreign Application Priority Data
May 20, 2005 (JP) 2005-147710
Jun. 21, 2005 (JP) 2005-181206
Jul. 26, 2005 (JP) 2005-216420
Oct. 21, 2005 (JP) 2005-307683

(51) Int. Cl. C08K 5/1535; C07D 307/12; C09K 3/00

(52) U.S. Cl. 524/113; 549/478; 252/182.11

(57) ABSTRACT

The present invention provides a (meth)acrylate containing a heterocyclic ring, which is a structure necessary for achieving physical properties required in many fields, and a hydrophilic group in the same monomer molecule and further provides a process for producing the same from industrially easily-available starting materials by industrially feasible reactions.

A (meth)acryloyloxytetrahydrofuran having a structure represented by the following general formula (1):

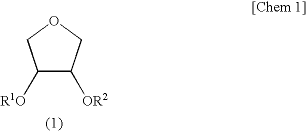

wherein R¹ and R² each is a hydrogen atom or a (meth)acryloyl group represented by the following general formula (2) and at least one of R¹ and R² is a (meth)acryloyl group represented by the general formula (2).

### TECHNICAL FIELD

The present invention relates to a (meth)acryloyloxytetrahydrofuran which is a novel compound and a process for producing the same in high yields and in high purity.

### BACKGROUND ART

In the case of producing vinyl copolymer resins, (meth)acrylate esters constitutes one of important monomer groups for copolymerization and are used in wide variety of applications. However, a desired performance is frequently not obtained by polymerization of a single monomer and, in those cases, a plurality of different (meth)acrylate ester monomers are mixed and copolymerized in order to obtain necessary physical properties. In particular, impartment of polarity to resins is one of the most important modification of the resins and monomers used for that purpose are (meth)acrylate ester monomers having a polar group. As representatives thereof, there may be mentioned linear hydroxyalkyl (meth)acrylates such as hydroxyethyl (meth)acrylate, hydroxypropyl (meth)acrylate, and hydroxybutyl (meth)acrylate. They have been widely employed owing to the situation that they are easily available in a large amount and in low costs since they can be easily produced from corresponding compounds having an epoxy skeleton or corresponding diols and (meth)acrylic acid. However, depending on applications, these hydroxy (meth)acrylates having a linear skeleton are not necessarily most appropriate in view of exhibiting desired properties. Rather, to the contrary, polarity is imparted but there arises a problem that a primarily necessary function is weakened or is not exhibited by the addition of the linear polar monomers.

For example, as a dental composition for fluoride release, it is disclosed that a composition containing a (meth)acrylic monomer having a heterocyclic ring is useful (Patent Document 1). However, in the case where it is intended to impart hydrophilicity to the composition, there is described in the specification that a conventional linear hydroxyalkyl (meth)acrylate can be used as a comonomer but since the hydroxyalkyl (meth)acrylate contains no heterocylic ring, it seems that function of releasing a fluoride is insufficient and thus the composition is not a suitable structure for the dental composition for fluoride release. If present, a (meth)acrylate ester having a hydroxyl group and exhibiting a high hydrophilicity might be useful for production of vinyl polymer resins requiring a heterocyclic ring.

In another application, a (meth)acrylate ester having a hydroxyl group is converted into a urethaneacrylate through a reaction with a diisocyanate and the product is used in combination with a (meth)acrylate having a heterocyclic ring.

For example, it is disclosed that, as a radiation-curable resin composition, a composition comprising a polyfunctional urethane (meth)acrylate (a component imparting viscosity to a resin) prepared from a hydroxy (meth)acrylate such as hydroxyethyl methacrylate and a mixture of a polyol and a diisocyanate and a component having a heterocyclic compound such as tetrahydrofurfuryl (meth)acrylate is preferred (Patent Document 2). This is because hardness of a cured film layer is not sufficiently increased and thus a desired performance is not achieved unless a heterocyclic compound is not added. Therefore, when a heterocyclic compound (meth)acrylate having a hydroxyl group is developed, it may serve many uses since production of a urethane acrylate having a heterocyclic structure is enabled.

Thus, a (meth)acrylate having an increased hydrophilicity owing to possession of both of a heterocyclic ring and a hydrophilic group in the same monomer is a compound whose development is expected in view of utilization of its high hydrophilicity and diversified capabilities of conversion into various compounds based on the hydrophilic group.

Furthermore, in the recent ArF photoresist field for semiconductors, (meth)acrylic resins have been employed as a main stream of resist materials but in the production of the photoresist resins for ArF, it is necessary to introduce a cyclic hydrocarbon structure such as an adamantane skeleton in order to enhance anti-etching performance (Non-Patent Document 1). However, on the other hand, since introduction of a large amount of hydrocarbon groups results in decrease in solubility of the resin to a developing liquid, it is necessary to modify the resin for improving solubility to aqueous developing liquid. Currently, for the purpose, there is used a resin to which a monomer having a polar group such as hydroxyadamantyl methacrylate is added. However, there are problems that the amount to be added should be increased owing to its insufficient hydrophilicity and the production of the resist resin totally cost high owing to its expensiveness. Therefore, also in this field, it has been desired to develop a (meth)acrylate monomer having a high hydrophilicity capable of efficiently imparting hydrophilicity to the resin.

On the other hand, with regard to a process for producing 3-(meth)acryloyloxy-4-hydroxytetrahydrofuran belonging to (meth)acryloyloxytetrahydrofurans, it is a current situation that no industrially advantageous process has not yet been found since the compound is a novel compound. In particular, simple application of conventional technologies may not dissolve a problem of contamination of di(meth)acryloyloxytetrahydrofuran in a considerable amount. Specifically, a conventional general method of extracting organic compounds includes post-treatment of an aqueous system after a reaction and subsequent recovery by extraction with an organic solvent capable of extracting the objective (meth)acrylate esters (see Patent Document 2). However, the content of di(meth)acryloyloxytetrahydrofuran cannot be reduced by this method.

Patent Document 1: JP-A-8-301718

Patent Document 2: JP-A-7-48422

Non-Patent Document 1: J. Photopolym. Sci. Technol., 9. 509 (1996).

Non-Patent Document 2: Tetrahedron 58 (2002) 5909.

### DISCLOSURE OF THE INVENTION

### Problems that the Invention is to Solve

An object of the invention is to provide a (meth)acrylate comprising a heterocyclic ring which is a structure necessary to achieve physical properties required in many fields and a hydrophilic group in the same monomer molecule and also to provide a process for producing the (meth)acrylate from an industrially easily available raw materials by industrially feasible reactions.

Moreover, according to the inventors' investigation, 3-(meth)acryloyloxy-4-hydroxytetrahydrofuran can be synthesized through selective monoesterification by reacting 3,4-dihydroxytetrahydrofuran as a raw material with a reagent such as (meth)acrylic acid, (meth)acryloyl chloride, or (meth)acrylic anhydride but not a little amount of di(meth)acryloyloxytetrahydrofuran is produced as a by-product. Furthermore, in the post-treatment after the reaction, at the time when an objective compound, 3-(meth)acryloyloxy-4-hydroxytetrahydrofuran is recovered by extraction, it is necessary to use an organic solvent capable of effectively extracting 3-(meth)acryloyloxy-4-hydroxytetrahydrofuran from an aqueous medium but almost whole amount of the concomitant di(meth)acryloyloxytetrahydrofuran might be also extracted when usual extraction is performed using such a solvent.

Accordingly, since the 3-(meth)acryloyloxy-4-hydroxytetrahydrofuran extracted such a conventional method contains a considerable amount of di(meth)acryloyloxytetrahydrofuran, there might be caused a serious problem that molecular weight cannot be controlled as intended during polymerization or a three-dimensional structure of the polymerized product may be different. This is attributable to two polymerizable functional groups contained in di(meth)acryloyloxytetrahydrofuran. Namely, when the contaminated di(meth)acryloyloxytetrahydrofuran participates in polymerization, a polymerization product having a structure crosslinked with two acryloyl group is formed and thus there is formed a polymerization product having a higher molecular weight as compared with the polymerization product derived from 3-(meth)acryloyloxy-4-hydroxytetrahydrofuran alone. Therefore, molecular weight distribution is broadened, which might cause physical properties different from those designed.

Thus, an object of the invention is to provide highly pure 3-(meth)acryloyloxy-4-hydroxytetrahydrofuran containing a small amount of di(meth)acryloyloxytetrahydrofuran which causes the crosslinking reaction during polymerization and also to provide a process for producing the objective compound by an industrially feasible procedure.

### Means for Solving the Problems

As a result of the extensive studies for solving the above problems, it has been found that a (meth)acryloyloxytetrahydrofuran represented by the general formula (1) has a structure containing a heterocyclic ring and a hydrophilic group in the same monomer molecule and actually, exhibits an extremely good solubility to aqueous solvents and a high hydrophilicity to water. Thus, the invention has been accomplished.

Furthermore, in the production of 3-(meth)acryloyloxy-4-hydroxytetrahydrofuran by (meth)acryloylation of 3,4-dihydroxytetrahydrofuran, it has been found that an extremely highly pure 3-(meth)acryloyloxy-4-hydroxytetrahydrofuran can be produced when a by-product, di(meth)acryloyloxytetrahydrofuran is removed by extraction with an extraction solvent containing at least water and a hydrocarbon after the (meth)acryloylation reaction. Thus, the invention has been accomplished.

Namely, the gist of the invention is as follows.

(1) A (meth)acryloyloxytetrahydrofuran having a structure represented by the following general formula (1):

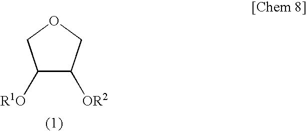

wherein R¹ and R² each is a hydrogen atom or a (meth)acryloyl group represented by the following general formula (2) and at least one of R¹ and R² is a (meth)acryloyl group represented by the general formula (2):

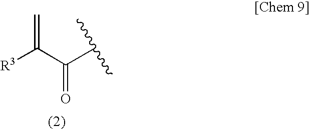

wherein R³ is a hydrogen atom or a methyl group and the wavy line represents a bonding site.

(2) A process for producing the (meth)acryloyloxytetrahydrofuran according to the above (1), which comprises reacting 3,4-dihydroxytetrahydrofuran represented by the following formula (3):

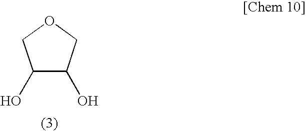

with a (meth)acryloyl halide in the presence of a basic substance.

(3) The process for producing the (meth)acryloyloxytetrahydrofuran according to the above (2), wherein the reaction is carried out further in the presence of an aprotic polar solvent miscible with water.

(4) The process for producing the (meth)acryloyloxytetrahydrofuran according to the above (3), wherein the aprotic polar solvent miscible with water is at least one solvent selected from ketones, ethers, nitriles, amides, and sulfoxides, each of which has 10 or less carbon atoms.

(5) The process for producing the (meth)acryloyloxytetrahydrofuran according to any one of the above (2) to (4), wherein a (meth)acryloyl halide having a purity of 85% by mol or more is used as the (meth)acryloyl halide of a raw material.

(6) The process for producing the (meth)acryloyloxytetrahydrofuran according to any one of the above (3) to (6), wherein a (meth)acryloyl halide containing a dimer of (meth)acryloyl halide in an amount of 15% by mol or less is used as the (meth)acryloyl halide of a raw material.

(7) The process for producing the (meth)acryloyloxytetrahydrofuran according to any one of the above (2) to (6), wherein the amount of water contained in 3,4-dihydroxytetrahydrofuran of a raw material is 10% by mol or less relative to 3,4-dihydroxytetrahydrofuran.

(8) A (meth)acryloyloxytetrahydrofuran composition, wherein each content of the compounds having structures represented by the following general formulae (4) and (5) in the above general formula (1) is 10% or less as an area ratio when analyzed by gel permeation chromatography with an RI detector:

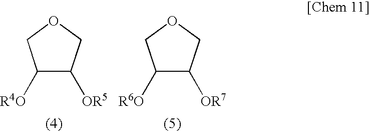

wherein R⁴ is a hydrogen atom or a (meth)acryloyl group represented by the above general formula (2) and R⁵, R⁶, and R⁷ each represents a group represented by the following general formula (6) or (7):

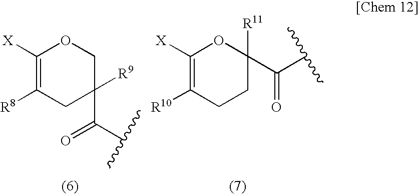

wherein R⁸, R⁹, R¹⁰, and R¹¹ each is a hydrogen atom or a methyl group, X represents a halogen atom, and the wavy line represents a bonding site.

(9) A process for producing a (meth)acryloyloxytetrahydrofuran, which comprises removing a di(meth)acryloyloxytetrahydrofuran by extraction with an extraction solvent containing water and a hydrocarbon solvent from a (meth)acryloyloxytetrahydrofuran composition containing a (meth)acryloyloxytetrahydrofuran having a structure represented by the following general formula (8) and a di(meth)acryloyloxytetrahydrofuran represented by the following general formula (9):

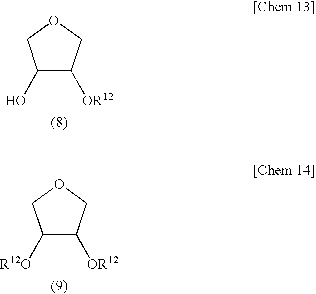

wherein R¹² is a (meth)acryloyl group represented by the above general formula (2).

(10) The process for producing a (meth)acryloyloxytetrahydrofuran according to the above (9), wherein an extraction solvent containing an aprotic polar solvent miscible with water is further used.

(11) The process for producing a (meth)acryloyloxytetrahydrofuran according to the above (9) or (10), wherein the (meth)acryloyloxytetrahydrofuran composition is obtained by reacting 3,4-dihydroxytetrahydrofuran represented by the above formula (3) with a (meth)acryloyl halide in the presence of a basic substance.

(12) The process for producing a (meth)acryloyloxytetrahydrofuran according to the above (11), wherein the reaction is carried out further in the presence of an aprotic polar solvent miscible with water.

(13) The process for producing a (meth)acryloyloxytetrahydrofuran according to any one of the above (10) to (12), wherein the content of the aprotic polar solvent miscible with water is 40% by weight or less relative to the weight of water.

(14) The process for producing a (meth)acryloyloxytetrahydrofuran according to any one of the above (10) to (13), wherein the aprotic polar solvent miscible with water is at least one solvent selected from ketones, ethers, nitrites, amides, and sulfoxides, each of which has 10 or less carbon atoms.

(15) A 3-(meth)acryloyloxy-4-hydroxytetrahydrofuran composition, wherein the abundance ratio of a (meth)acryloyloxytetrahydrofuran represented by the above general formula (8) to a di(meth)acryloyloxytetrahydrofuran represented by the above general formula (9) is 97/3 or more as an area ratio when analyzed by gas chromatography with an FID detector.

(16) A resist resin composition comprising a (meth)acryloyloxytetrahydrofuran represented by the above general formula (1) as a constitutional component.

(17) The resist resin composition according to the above (16), which comprises, as a constitutional component, an acid-dissociating monomer as a copolymer composition.

### ADVANTAGE OF THE INVENTION

The (meth)acryloyloxytetrahydrofuran of the invention has a structure containing a heterocyclic ring and a hydrophilic group in the same monomer molecule and actually exhibits an extremely good solubility to aqueous solvents and thus a high hydrophilicity to water, so that it is possible to utilize it for the purpose of modification to impart hydrophilicity to various (meth)acrylic resins. Examples of usable fields include resins for resists such as color resists and semiconductor resists, resins for medical materials such as dental materials, resins for paints and coatings, resins for adhesives, and resins for textile treatment. Furthermore, in the recent resist field, a technical development by an immersion method has been actively performed in an image-forming technology by ArF laser and necessity of a topcoat resin capable of being dissolved in an alkaline developing fluid has been increased. Thus, in view of the characteristics of the compound of the invention, it might be also possible to apply it to this application.

Additionally, according to the production process of the invention, it is possible to produce the polymerizable monomer rich in hydrophilicity in good yields by industrially simple and convenient operations.

Since the highly pure 3-(meth)acryloyloxy-4-hydroxytetrahydrofuran of the invention exhibits an extremely good solubility owing to its high hydrophilicity, it can efficiently impart hydrophilicity in a small copolymerization ratio when converted into a polymer. Moreover, since the content of di(meth)acryloyloxytetrahydrofuran which makes control of polymerization degree difficult during polymerization is extremely low, a polymer with a predetermined molecular weight distribution can be obtained, so that it becomes possible to achieve objective physical properties. Furthermore, according to the production process of the invention, it becomes possible to produce 3-(meth)acryloyloxy-4-hydroxytetrahydrofuran in good yields by industrially simple and conventional operations.

### BEST MODE FOR CARRYING OUT THE INVENTION

The following will describe the invention in detail.

### <(Meth)acryloyloxytetrahydrofuran>

The polymerizable monomer of the invention has a structure represented by the following general formula (1):

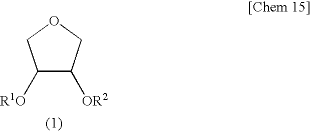

wherein R¹ and R² each is a hydrogen atom or a (meth)acryloyl group represented by the following general formula (2) and at least one of R¹ and R² is a (meth)acryloyl group represented by the general formula (2):

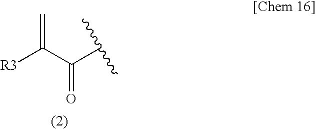

wherein R³ is a hydrogen atom or a methyl group and the wavy line represents a bonding site.

Namely, it is a structure wherein at least one of two hydroxyl groups of 3,4-dihydroxytetrahydrofuran (R¹ and R² each is a hydrogen atom in the formula (1)) is converted into a (meth)acrylate ester group. It was found that these compounds have a remarkably excellent hydrophilicity as compared with compounds wherein a (meth)acrylate ester is merely bonded to a hydrocarbon and compounds wherein only one (meth)acrylate ester group as an oxygen-functional group is condensed to compounds having a tetrahydrofuran ring.

Particularly, it was found that 3-(meth)acryloyloxy-4-hydroxytetrahydrofuran, which is a compound where one hydrogen and one (meth)acrylate ester group are condensed to R¹ and R² in the general formula (1), has an extremely high hydrophilicity and exhibits an excellent solubility to aqueous solvents. It is presumed that the whole molecule is apt to be solvated with water molecule and thus the solubility is increased since an OH group and a five-membered cyclic ether structure both showing hydrophilicity as well as an ester group are positioned in the molecule without deviation. On the other hand, since the compound also has a sufficient solubility to organic solvents, it has large advantages in handling and operation thereof, e.g., easy handling of the monomer itself, broad options in the selection of the solvent to be used for the polymerization, and the like.

### 3-(Meth)acryloyloxy-4-hydroxytetrahydrofuran

3-(Meth)acryloyloxy-4-hydroxytetrahydrofuran of the invention having an increased purity is represented by the structure represented by the following formula (8):

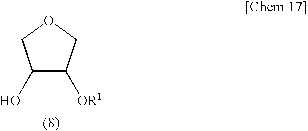

wherein R¹ is a (meth)acryloyl group represented by the following general formula (2),

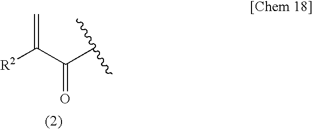

wherein R² is a hydrogen atom or a methyl group and the wavy line represents a bonding site.

Furthermore, 3-(meth)acryloyloxy-4-hydroxytetrahydrofuran according to the invention has a high purity and, in a typical case, the abundance ratio of 3-(meth)acryloyloxy-4-hydroxytetrahydrofuran to di(meth)acryloyloxytetrahydrofuran is 97/3 or more as an area ratio when analyzed by gas chromatography with an FID detector.

The following will describe a process for producing a (meth)acryloyloxytetrahydrofuran but 3-(meth)acryloyloxy-4-hydroxytetrahydrofuran is one example of the (meth)acryloyloxytetrahydrofuran and hence 3-(meth)acryloyloxy-4-hydroxytetrahydrofuran can be produced by the following production process without particular limitation.

<Process for Producing (meth)acryloyloxytetrahydrofuran>

In the production of the compound of the invention, (meth)acryloyloxytetrahydrofuran, particularly its production route is not limited and any production processes can be employed. In particular, a process using erythritol as a raw material is preferred since erythritol is available in low costs and in an industrial scale. In this case, either of a process of first esterifying erythritol and subsequently cyclizing it into (1) or a process of first cyclizing it into 3,4-dihydroxytetrahydrofuran (in the above (1), R¹ and R² each is a hydrogen atom and the both hydroxyl group has a cis-configuration in the structure in this case) and subsequently (meth)acrylating only necessary number of the hydroxyl group(s) can be arbitrarily used.

The second step of the (meth)acrylation reaction in the process via 3,4-dihydroxytetrahydrofuran can be arbitrarily selected. As representative methods, there may be suitably employed a method of esterifying the hydroxyl group using a (meth)acryloyl halide or (meth)acrylic anhydride, an ester-exchange reaction using an ester of a lower alcohol of (meth)acrylic acid, a direct esterification reaction of dehydrative condensation of (meth)acrylic acid with erythritan, and the like.

Moreover, for the purpose of producing a mono(meta)acrylate of 3,4-dihydroxytetrahydrofuran, processes for selectively producing the mono(meta)acrylate can be employed without particular limitation, for example, a process of first protecting one hydroxyl group of 3,4-dihydroxytetrahydrofuran, (meth)acrylating another hydroxyl group, and subsequently performing deprotection, a process of modifying two hydroxyl groups into a carbonate structure, selectively converting only one group into a (meth)acrylate group nucleophilically, and finally performing post-treatment with water, and the like.

As the compounds of the invention and reagents, (meth)acrylate compounds rich in polymerizability are used. A polymerization inhibitor may be employed so that polymerization does not proceed during the reaction and storage. Examples of the polymerization inhibitor include hydroquinones such as p-benzoquinone, hydroquinone, hydroquinone monomethyl ether, t-butylcatechol, and 2,5-diphenyl-p-benzoquinone, N-oxy radicals such as tetramethylpiperidinyl-N-oxy radical (TEMPO), phenothiazine, diphenylamine, phenyl-β-naphthylamine, nitrosobenzene, picric acid, molecular oxygen, sulfur, copper(II) chloride, and the like.

The amount of the polymerization inhibitor to be used is usually 10 ppm or more, preferably 50 ppm or more as a lower limit and usually 10,000 ppm or less, preferably 1,000 ppm or less as an upper limit relative to 3,4-dihydroxytetrahydrofuran or the compound of the general formula (1) as a product.

The following will describe employable reaction conditions on the representative esterification reaction of 3,4-dihydroxytetrahydrofuran.

<Process for Producing (meth)acryloyloxytetrahydrofuran; Ester-Exchange Method>

In the case where 3,4-dihydroxytetrahydrofuran is (meth)acrylated by an ester-exchange reaction, a compound usable as a (meth)acrylating agent is a lower alcohol ester of (meth)acrylic acid. As the lower alcohol, a C1 to C4 aliphatic alcohol is preferred and the number of the alcohol residues is selected from 1 to 3. Particularly preferred are methyl ester, ethyl ester, n-propyl ester, and i-propyl ester of (meth)acrylic acid.

The amount of the (meth)acrylate ester to be used is usually 0.1 molar equivalent or more, preferably 0.2 molar equivalent or more, more preferably 0.5 molar equivalent or more as a lower limit and usually 20 molar equivalents or less, preferably 10 molar equivalents or less, more preferably 5 molar equivalents or less as an upper limit relative to moles of 3,4-dihydroxytetrahydrofuran as a raw material. However, in the case where the diester of the above general formula (1) (wherein both of R¹ and R² are (meth)acryl esters), the amount of the (meth)acrylate ester to be used can be large excess to erythritan and, in an extreme case, it may be 50 molar equivalents or more.

The method of adding the (meth)acrylate ester is not particularly limited and both methods of performing the reaction with adding the whole amount thereof to 3,4-dihydroxytetrahydrofuran at the time when reactants are charged and adding the ester portionwise in the course of the reaction are employable.

The reaction can be carried out both with and without a solvent. In the case of using a solvent, the solvent to be used is not particularly limited and there may be suitably used aromatic solvents such as toluene and xylene, aliphatic hydrocarbon solvents such as hexane and heptane, ethereal solvents such as diethyl ether, tetrahydrofuran, monoethylene glycol dimethyl ether, and diethylene glycol dimethyl ether, ketone-based solvents such as acetone, methyl ethyl ketone, and methyl isobutyl ketone, ester-based solvents such as ethyl acetate, butyl acetate, and γ-butyrolactone, amide-based solvents such as dimethylformamide, dimethylacetamide, and N-methylpyrrolidone, and the like. These solvents may be used singly or may be used as a mixture of any two or more solvents.

In the case of using a solvent, the amount thereof is so that the concentration of 3,4-dihydroxytetrahydrofuran is usually 0.1% by weight or more, preferably 1% by weight or more as a lower limit and usually 80% by weight or less, preferably 50% by weight or less as an upper limit although the upper limit is not particularly limited.

The ester-exchange reaction is usually carried out in the presence of a catalyst. Catalysts generally usable in ester-exchange reactions are applicable and examples thereof include transition metal compounds such as titanium tetraisopropoxide, alcolates of alkali metals and alkaline earth metals, such as sodium methoxide, alkoxides of aluminum, such as aluminum triisopropoxide, hydroxides of alkali metals and alkaline earth metals, such as lithium hydroxide and sodium hydroxide, tin compounds such as dibutyltin oxide and dioctyltin oxide, and the like.

The amount of these catalysts to be used is usually 0.01% by mol or more, preferably 0.1% by mol or more, more preferably 0.5% by mol or more as a lower limit and usually 50% by mol or less, preferably 20% by mol or less, more preferably 10% by mol or less as an upper limit relative to moles of 3,4-dihydroxytetrahydrofuran as a raw material.

The reaction is preferably carried out in a reactor fitted with a usual stirring apparatus. In addition, the reaction may be carried out under removing alcohol generated during the reaction with moving equilibrium to the generating system. On this occasion, in the case where the (meth)acrylate ester used as a reagent is removed from the system by azeotrope formation thereof with the alcohol, the reaction may be carried out with sequential addition of the (meth)acrylate ester as needed.

With regard to the reaction temperature, the reaction is preferably carried out under heating so as to obtain a sufficient reaction rate. Specifically, the reaction is carried out in the range of usually −10° C. or higher, preferably 0° or higher as a lower limit and usually 200° C. or lower, preferably 150° C. or lower as an upper limit.

The reaction time is arbitrarily selected. Since the alcohol is formed as the reaction proceeds, the reaction is preferably continued until a predetermined amount of alcohol is formed. The common reaction time is usually 10 minutes or more, preferably 30 minutes or more as a lower limit and usually 50 hours or less, preferably 30 hours or less as an upper limit although the upper limit is not particularly limited.

<Process for Producing (meth)acryloyloxytetrahydrofurn; (meth)acryloyl halide Process, (meth)acrylic anhydride Process>

3,4-Dihydroxytetrahydrofuran can be (meth)acrylated using (meth)acryloyl halide or (meth)acrylic anhydride as a (meth)acrylating agent. In that case, compounds usable as the (meth)acryloyl halide are chloride, bromide, and iodide of (meth)acrylic acid.

The amount of the (meth)acryloyl halide or (meth)acrylic anhydride to be used is usually 0.01 molar equivalent or more, preferably 0.05 molar equivalent or more, more preferably 0.1 molar equivalent or more as a lower limit and usually 20 molar equivalents or less, preferably 10 molar equivalents or less, more preferably 5 molar equivalents or less as an upper limit relative to moles of 3,4-dihydroxytetrahydrofuran as a raw material.

With regard to the method of adding the (meth)acryloyl halide or (meth)acrylic anhydride, the addition method is not particularly limited as far as it is avoided to bring the (meth)acrylating agent into contact with a basic substance for a long time before the reaction. For example, 3,4-dihydroxytetrahydrofuran and (meth)acryloyl halide or (meth)acrylic anhydride may be simultaneously charged into a reactor and then a basic substance may be added, or (meth)acryloyl halide or (meth)acrylic anhydride may be added dropwise to a solution of a basic substance and 3,4-dihydroxytetrahydrofuran having been charged into a reactor beforehand to thereby effect the reaction. In the case of producing mono(meth)acrylate of 3,4-dihydroxytetrahydrofuran, the latter addition method is preferably adopted in view of reducing a by-product.

In the case where the reaction is carried out using (meth)acryloyl halide or (meth)acrylic anhydride, it is necessary to control the water content properly. When moisture is present in the system, the reagent cannot be efficiently utilized since the moisture may reacts with the (meth)acryloyl halide or (meth)acrylic anhydride. The substrate to be used in the invention, e.g., 3,4-dihydroxytetrahydrofuran is easily miscible with water but lesser water content in the substrate is more preferred. Specifically, the water content is 10% by mol or less, preferably 5% by mol or less, more preferably 1% by mol or less relative to 3,4-dihydroxytetrahydrofuran.

The reaction can be carried out either with no solvent or with a solvent. In the case of using a solvent, there may be suitably used aromatic hydrocarbon solvents such as toluene and xylene, aliphatic hydrocarbon solvents such as hexane and heptane, ethereal solvents such as diethyl ether, tetrahydrofuran, monoethylene glycol dimethyl ether, and diethylene glycol dimethyl ether, ketone-based solvents such as acetone, methyl ethyl ketone, and methyl isopropyl ketone, ester-based solvent such as ethyl acetate, butyl acetate, and γ-butyrolactone, amide-based solvents such as dimethylformamide, dimethylacetamide, and N-methylpyrrolidone, and the like. These solvents may be used singly or may be used as a mixture of any two or more solvents.

In the case of using a solvent, the amount is such an amount that the concentration of 3,4-dihydroxytetrahydrofuran as a raw material is usually 0.1% by weight or more, preferably 1% by weight or more as a lower limit and usually 80% by weight or less, preferably 50% by weight or less as an upper limit although the upper limit is not particularly limited.

The (meth)acrylation reaction with (meth)acryloyl halide or (meth)acrylic anhydride is usually carried out in the presence of a basic substance. As usable basic substances, it is possible to use metal hydroxides such as sodium hydroxide and barium hydroxide, metal carbonates such as sodium carbonate and potassium carbonate, metal phosphates and hydrogen phosphates such as monosodium phosphate and potassium phosphate, basic ion-exchange resins, organic tertiary amines such as triethylamine and tributylamine, aromatic amines such as pyridine, and the like. Of these, pyridine, triethylamine, and potassium carbonate are suitably used.

The amount of the basic substance to be used is usually 0.1 molar equivalent or more, preferably 0.5 molar equivalent or more, more preferably 1.0 molar equivalent or more as a lower limit and usually 10 molar equivalents or less, preferably 5 molar equivalents or less, more preferably 2 molar equivalents or less as an upper limit relative to (meth)acryloyl halide or (meth)acrylic anhydride to be used.

The reaction is preferably carried out in a reactor fitted with a usual stirring apparatus.

With regard to the reaction temperature to be adopted, the reaction is carried out in the range of usually −50° C. or higher, preferably −20° or higher as a lower limit and usually 100° C. or lower, preferably 70° C. or lower as an upper limit. In the case of producing mono(meth)acrylate of 3,4-dihydroxytetrahydrofuran, it is preferred to set the upper limit at a low temperature and the reaction is carried out in the range of usually 50° C. or lower, preferably 30° C. or lower as an upper limit.

The reaction time is arbitrarily selected. Including the time for dropwise addition of the reagent(s), the common reaction time is usually 10 minutes or more, preferably 30 minutes or more as a lower limit and usually 20 hours or less, preferably 10 hours or less as an upper limit although the upper limit is not particularly limited.

In the case where the (meth)acrylation reaction is carried out with (meth)acryloyl halide, it is necessary to pay attention on the purity of the (meth)acryloyl halide to be used, depending on the substrate. A (meth)acryloyl halide has a characteristic that it is dimerized with time to form an impurity having a structure represented by the following formula (10) or (11), thereby the purity being decreased, as described in Chimia, 1985, vol. 39, p. 19-20.

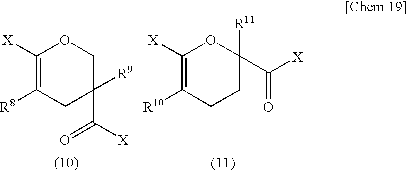

In the formulae (10) and (11), R⁸, R⁹, R¹⁰, and R¹¹ each represents a hydrogen atom or a methyl group and X represents a halogen atom, preferably a chlorine atom.

Usually, in the case where a hydroxyl group having a sterically somewhat hindered environment is esterified using an acid halide, the acid halide portion of the dimer is sterically hindered, so that the dimer has a lower activity than the monomer acid halide has and hence may not be involved in the reaction. However, for example, in the case where 3,4-dihydroxytetrahydrofuran having a cyclic structure is used as a substrate, the circumstance of the hydroxyl group is relatively sterically not hindered, so that it has a high reactivity toward the acid halide. Therefore, when 3,4-dihydroxytetrahydrofuran is intended to be (meth)acrylated using an acid halide containing a large amount of the dimer having the above structure, there arise a problem that by-products having the following structures (4) and (5) which is formed by the reaction with the dimer are produced in addition to the product with the acid halide.

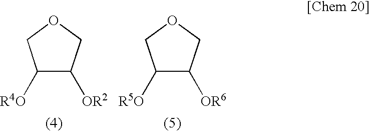

In the formulae (4) and (5), R² is a hydrogen atom or a (meth)acryloyl group represented by the above general formula (2) and R⁴, R⁵, and R⁶ each represents a group represented by the following general formula (18) or (19).

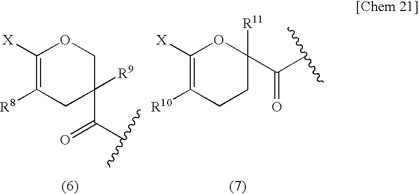

In the formulae (6) and (7), R⁷, R⁸, R⁹, and R¹⁰ each represents a hydrogen atom or a methyl group and X represents a halogen atom, preferably a chlorine atom. The wavy line represents a bonding site. For the reasons mentioned above, in the case where a hydroxyl group such as 3,4-dihydroxytetrahydrofuran which is relatively sterically not hindered is (meth)acrylated with a (meth)acryloyl halide, it is necessary to use a reagent which is the (meth)acryloyl halide having a high purity. Specifically, there is used a reagent wherein the purity of the (meth)acryloyl halide is usually 80% by mol or more, preferably 85% by mol or more, more preferably 90% by mol or more, particularly preferably 95% by mol or more.

Moreover, the esterification is preferably carried out using a (meth)acryloyl halide wherein the content of the dimers of (10) and (11) is usually 20% by mol or less, preferably 15% by mol or less, more preferably 10% by mol or less, particularly preferably 5% by mol or less.

The method for enhancing the purity of the (meth)acryloyl halide is not particularly limited but distillation utilizing the difference between boiling points of the acid halide and the diners is convenient and preferred. As the method of distillation, simple distillation, rectification, thin-film distillation, and the like can be adopted without limitation.

When 3,4-dihydroxytetrahydrofuran is (meth)acrylated using the thus purified (meth)acryloyl halide, (meth)acryloyloxytetrahydrofuran represented by the above formula (1) containing a small amount of the by-products represented by the structural formulae (4) and (5) can be obtained. In this connection, when the compound represented by the above formula (1) contains (4) and (5) as impurities, there arises production problems that difference in polymerization rate may occur during the subsequent polymerization and an insoluble matter may be formed as well as the performance of the resin itself may be affected, so that the case is not preferred. The compound of the invention represented by the above formula (1) is a compound represented by the general formula (1) wherein the contents of the compounds having structures of the general formulae (4) and (5) each is 10% by mol or less as an area ratio when analyzed by gel permeation chromatography with an RI detector.

<Process for Producing (meth)acryloyloxytetrahydrofuran; Direct Dehydration Process>

In the case of esterification with (meth)acrylic acid, the reaction promptly proceeds in the presence of a dehydrative condensation agent. As the condensation agent, any one can be used without particular limitation as far as it is known as a condensation agent for esterification and, for example, N,N′-dicyclohexylcarbodiimide, 2-chloro-1,3-dimethylimidazolium chloride, propanephosphonic anhydride, and the like are suitably used. On this occasion, an organic basic substance such as pyridine, 4-dimethylaminopyridine, or triethylamine may be used in combination. The reaction temperature usually adopted in the reaction is usually −20° C., preferably −10° C. as a lower limit and usually 150° C., preferably 100° C. as an upper limit.

With regard to the amount of the dehydrative condensation agent, use of an equivalent amount to 3,4-dihydroxytetrahydrofuran as a substrate is theoretically sufficient but an excess amount may be used. Preferably, the amount is 1.0 molar equivalent or more, more preferably 1.1 molar equivalents or more.

In the case of using no dehydrative condensation agent, (meth)acrylic acid and 3,4-dihydroxytetrahydrofuran are reacted in the presence of an acid while water formed is removed by distillation.

As the acid to be used, any acid used for usual esterification reaction can be used without particular limitation. Examples thereof include inorganic acids such as sulfuric acid and hydrochloric acid, organic sulfonic acids such as p-toluenesulfonic acid, methanesulfonic acid, and camphorsulfonic acid, acid-type ion-exchange resins, Lewis acids such as boron trifluoride-ether complex, water-soluble Lewis acids such as lanthanide triflate, and the like. These acids may be used singly or as a mixture of two or more of any acids.

The lower limit of the amount of the acid to be used is 0.001% by mol or more, preferably 0.01% by mol or more, more preferably 0.1% by mol or more relative to 3,4-dihydroxytetrahydrofuran. On the other hand, the upper limit is not limited but is 10 molar equivalents or less, preferably 1 molar equivalent or less.

The reaction can be carried out either with no solvent or with a solvent. In the case of using a solvent, there may be suitably used aromatic hydrocarbon solvents such as toluene and xylene, aliphatic hydrocarbon solvents such as hexane and heptane, ethereal solvents such as diethyl ether, tetrahydrofuran, monoethylene glycol dimethyl ether, and diethylene glycol dimethyl ether, halogenated solvents such as methylene chloride, chloroform, and carbon tetrachloride, and the like. These solvents may be used singly or may be used as a mixture of any two or more solvents.

In the case of using a solvent, the amount is such an amount that the concentration of 3,4-dihydroxytetrahydrofuran as a raw material is usually 0.1% by weight or more, preferably 1% by weight or more as a lower limit and usually 80% by weight or less, preferably 50% by weight or less as an upper limit although the upper limit is not particularly limited.

The reaction is usually carried out at a temperature of the boiling point of the solvent used or higher and the reaction is carried out while water formed is removed by distillation.

The reaction time is arbitrarily selected and the end point of the reaction can be recognized by measuring the amount of water formed. Including the time for dropwise addition of the reagent(s), the common reaction time is usually 10 minutes or more, preferably 30 minutes or more as a lower limit and usually 20 hours or less, preferably 10 hours or less as an upper limit although the upper limit is not particularly limited.

### <Method for Quenching and Concentrating Reaction Mixture>

As operations after the synthesis in the above manner, in 3-(meth)acryloyloxy-4-hydroxytetrahydrofuran, the following method is preferably used. Specifically, the reaction mixture is quenched and, in the case where a solvent is used in the reaction, the solvent is concentrated as needed. In the case where an inorganic salt is formed in the reaction, after the reaction reagent is quenched by adding a small amount of water, the inorganic salt is filtrated and then a predetermined amount of water may be added thereto.

The water to be added may contain an acid or an alkali depending on the necessity for quenching. As the acid to be contained at that time, inorganic acids such as sulfuric acid, nitric acid, hydrochloric acid, and phosphoric acid may be exemplified. As the alkali, alkali metal hydroxides such as sodium hydroxide and potassium hydroxide, metal carbonates such as sodium carbonate and sodium hydrogen carbonate, alkyl metal alcolates such as sodium ethoxide, and the like can be used.

The amount of the water to be added is 0.1 weight equivalent or more, preferably 0.5 weight equivalent or more, relative to 3,4-dihydroxytetrahydrofuran, as a lower limit and 50 weight equivalents or less, preferably 20 weight equivalents or less in view of volume efficiency of the reactor to be used although the upper limit is not particularly limited.

After water was added, the reaction solvent is concentrated as needed. At the concentration, a polymerization inhibitor may be added as needed. Examples of the polymerization inhibitor usable include hydroquinones such as p-benzoquinone, hydroquinone, hydroquinone monomethyl ether, t-butylcatechol, and 2,5-diphenyl-p-benzoquinone, N-oxy radicals such as tetramethylpiperidinyl-N-oxy radical (TEMPO), phenothiazine, diphenylamine, phenyl-β-naphthylamine, nitrosobenzene, picric acid, molecular oxygen, sulfur, copper(II) chloride, and the like.

The amount of the polymerization inhibitor to be used is usually 10 ppm or more, preferably 50 ppm or more as a lower limit and usually 10,000 ppm or less, preferably 1,000 ppm or less as an upper limit relative to the weight of the compound of the general formula (1) as a product.

In the concentration of the solvent, either of normal pressure and reduced pressure can be adopted but reduced pressure wherein a temperature necessary for the concentration is low is preferred since the objective compound is polymerizable and thermal stability is not high.

The degree of concentration is not particularly limited in the case of using a solvent immiscible in water but, in view of the reactor efficiency in the production, the upper limit is 20 equivalents or less, preferably 10 equivalents or less as a weight ratio of the remaining solvent to the objective compound. The lower limit may be complete removal by distillation. However, in the case where whole amount or partial amount of the reaction solvent is used as a solvent for extraction and purification to be subsequently performed, the limits are not applicable.

In the case where an aprotic polar solvent miscible with water is used in the (meth)acryloylation reaction, the aprotic polar solvent remains in the concentrated solution even after performing the concentration but the weight ratio of the remaining aprotic polar solvent to water becomes important at that time. Namely, since purification efficiency can be enhanced and a highly pure objective compound can be obtained when the remaining amount of the aprotic polar solvent miscible with water decreases, it is desirable to reduce the remaining amount of the aprotic polar solvent miscible with water by performing concentration as far as possible. Specifically, the amount is an equivalent amount or less, preferably 40% or less, more preferably 20% or less relative to the weight of water to be present in the system at extraction.

### <Extraction Method>

After the concentration of the reaction solvent, the extraction operation of the invention is performed. In the reaction of (meth)acryloylation of 3,4-dihydroxytetrahydrofuran, di(meth)acyloyloxytetrahydrofuran is usually produced as a by-product in addition to the objective compound, 3-(meth)acryloyloxy-4-hydroxytetrahydrofuran. The ratio is usually about 70/30 or more and 97/3 or less as an area ratio of 3-(meth)acryloyloxy-4-hydroxytetrahydrofuran/di(meth)acryloyloxytetrahydrofuran when analyzed by gas chromatography with an FID detector.

In the invention, the di(meth)acryloyloxytetrahydrofuran produced as a by-product is removed by extraction. At that time, the composition of the extract solution is important and an effective removal of the by-product becomes possible by adjusting the composition to the composition of the invention, so that it becomes possible to produce a highly pure 3-(meth)acryloyloxy-4-hydroxytetrahydrofuran of the invention.

The liquid composition at the time of extraction in the invention is a composition containing at least water and a hydrocarbon in addition to the objective compound and the by-product. When extracted with the composition, 3-(meth)acryloyloxy-4-hydroxytetrahydrofuran is mainly distributed into an aqueous layer and di(meth)acryloyloxytetrahydrofuran is mainly distributed into a hydrocarbon layer, so that di(meth)acryloyloxytetrahydrofuran can be efficiently removed.

The hydrocarbon solvent usable at the time of extraction can be freely selected but the solvent having 10 or less carbon atoms is preferred in view of easy handling. Specifically, there may be mentioned linear hydrocarbon solvents such as n-hexane and n-heptane, cyclic hydrocarbon solvents such as cyclohexane, aromatic hydrocarbon solvents such as toluene and xylene, and the like. Of these, linear hydrocarbon solvents exhibiting a low solubility to 3-(meth)acryloyloxy-4-hydroxytetrahydrofuran are preferred and more specifically, n-hexane, n-heptane, cyclohexane, and toluene are preferred. The amount of the hydrocarbon solvent to be used is not basically limited but, in order to attain an effective extraction efficiency of di(meth)acryloyloxytetrahydrofuran, the lower limit is 0.01 weight equivalent or more, preferably 0.1 weight equivalent or less, more preferably 0.5 weight equivalent or more, particularly preferably 1 weight equivalent or more relative to the weight of the aqueous layer to be extracted. The upper limit is 100 weight equivalents or less, preferably 50 weight equivalents or less, more preferably 20 weight equivalents or more, particularly preferably 10 weight equivalents or more for the economical reason. The extraction operation is preferably performed with dividing the amount of the hydrocarbon solvent within the above range into several portions.

When the amount of water at the extraction is too small, loss of the objective compound 3-(meth)acryloyloxy-4-hydroxytetrahydrofuran increases. On the other hand, when the amount is too large, the efficiency at the time of extraction of the objective compound 3-(meth)acryloyloxy-4-hydroxytetrahydrofuran becomes worse after the removal of di(meth)acryloyloxytetrahydrofuran with the hydrocarbon solvent. Therefore, care should be taken to the amount of water. With regard to the preferable amount of water, the lower limit is 0.5 weight equivalent or more, preferably 1.0 weight equivalent or more, more preferably 2.0 weight equivalents or more, particularly preferably 3.0 weight equivalents or more relative to the weight of 3-(meth)acryloyloxy-4-hydroxytetrahydrofuran as the reaction product. The upper limit is usually 100 weight equivalents or less, preferably 50 weight equivalents or less, more preferably 20 weight equivalents or less, particularly preferably 10 weight equivalents or less. When the amount does not fall within the above range, addition of water or removal by distillation is performed prior to the extraction.

In addition to the hydrocarbon solvent to be used at the extraction, a polar solvent immiscible with water may be added. In that case, efficiency of removing 3,4-di(meth)acryloyloxytetrahydrofuran can be enhanced.

Examples of the polar solvent immiscible with water includes ethereal solvents such as diethyl ether and diisopropyl ether, ketone-based solvents such as methyl isobutyl ketone, ester-based solvents such as ethyl acetate and butyl acetate, and the like. Of these, ketone-based solvents and ester-based solvents are preferred in view of the efficiency of removing di(meth)acryloyloxytetrahydrofuran.

A plurality of these solvents may be used in combination as a mixture with a hydrocarbon solvent. With regard to the amount of the polar solvent immiscible with water, when the amount is too large relative to the hydrocarbon solvent, the objective compound 3-(meth)acryloyloxy-4-hydroxytetrahydrofuran is also extracted, so that care should be taken. The upper limit is 10 times or less, preferably 5 times or less, more preferably 3 times or less, particularly preferably 1 time or less as a volume ratio to the hydrocarbon solvent to be used. The lower limit is not particularly limited and the polar solvent may not be used.

In the case where the polar solvent miscible with water is used in the reaction, the composition of the liquid for extraction after solvent concentration is adjusted to the following composition in order to remove di(meth)acryloyloxytetrahydrofuran by extraction efficiently. Namely, the content of the aprotic non-polar solvent miscible with water is reduced to an equivalent amount or less, preferably 40% or less, more preferably 20% or less relative to the weight of water also present in the system.

The extraction operation can be performed at an arbitrary temperature but it is probable that the operation may be impossible at a temperature equal to or higher than the boiling point of the hydrocarbon solvent to be used and at a temperature equal to or lower than the melting point thereof. Therefore, the upper limit is usually 100° C. or lower, preferably 50° C. or lower and the lower limit is 0° C. or higher, preferably 10° C. or higher.

In the aqueous layer after the extraction, di(meth)acryloyloxytetrahydrofuran is removed and 3-(meth)acryloyloxy-4-hydroxytetrahydrofuran having an increased purity is present. This compound is extracted with a polar solvent immiscible with water. As the solvent to be used in the extraction, there may be mentioned ethereal solvents such as diethyl ether and diisopropyl ether, ketone-based solvents such as methyl isobutyl ketone, ester-based solvents such as ethyl acetate and butyl acetate, and the like. They may be used singly or as a combination of two or more thereof.

The amount of the solvent to be used in the extraction is 0.1 equivalent or more, preferably 1 equivalent or more to the weight of 3-(meth)acryloyloxy-4-hydroxytetrahydrofuran to be extracted as a lower limit. The upper limit is not particularly limited but, in view of the volume efficiency of the facility for production, is 100 equivalents or less, preferably 50 equivalents or less. When the extracted solution is concentrated, objective 3-(meth)acryloyloxy-4-hydroxytetrahydrofuran can be obtained.

In thus obtained 3-(meth)acryloyloxy-4-hydroxytetrahydrofuran, the content of di(meth)acryloyloxytetrahydrofuran is extremely low and, in typical cases, the amount is 97/3 or more, preferably 98/2 or more, more preferably 99.5/0.5 or more as an area ratio when the abundance ratio of 3-(meth)acryloyloxy-4-hydroxytetrahydrofuran to di(meth)acryloyloxytetrahydrofuran is analyzed by gas chromatography with an FID detector. Moreover, the upper limit is not particularly limited since the purity of the resulting compound increases as the upper limit is heightened. The compound has a characteristic that formation of high-molecular-weight compounds is reduced when used as a raw material for polymerization.

### <Purification Method>

For example, the purification of the compounds represented by the general formula (1) produced by the above-mentioned reactions can be performed without particular limitation. For example, a distillation method, a recrystallization method, an extraction-washing method, and the like may be applied. In the case of distillation, the mode may be any one selected from simple distillation, rectification, thin-film distillation, molecular distillation, and the like.

### <Storage Method>

The 3-(meth)acryloyloxy-4-hydroxytetrahydrofuran obtained by the above-mentioned extraction operation is preferably stored at room temperature or a lower temperature since it is polymerizable. Furthermore, it is more preferred to store it in a refrigerator.

### <Applications>

The (meth)acryloyloxytetrahydrofuran obtained by the invention can be widely utilized as a raw material for vinyl polymerization resins in a variety of fields such as electronic parts materials, optical applications, recording media, various curing agents, and medical materials.

### EXAMPLES

The following will describe the invention further in detail with reference to Examples but the invention is not limited to the following Examples unless it exceeds the gist.

### <Analysis of Purity by Gas Chromatography>

Column: TG-1 manufactured by GL science, 0.25 mm, 30 m, 0.25 μm

Carrier gas: helium

Detector: FID

Injection temperature: 250° C.

Column chamber temperature: initial temperature 50° C. (kept for 5 min)

Temperature-elevating rate: 10° C./min

Final temperature: 250° C. (kept for 20 min)

Injected amount: 0.2 μL

### <Analytical Conditions for Gel Permeation Chromatography (Hereinafter Abbreviated as GPC)>

Column: Tosoh TSK-GEL G2000HXL
7.8 mm(ID)×300 mm(L)×two columns

Moving phase: THF 1 mL/min

Detector: RI

Column chamber temperature: 40° C.

Injected amount: 50 μL (0.1% THF solution)

### <Compound Names>

The following common names are used in Examples.

Erythritan: cis-3,4-dihydroxytetrahydrofuran

Erythritan monomethacrylate: 3-methacryloyloxy-4-hydroxytetrahydrofuran

Erythritan dimethacrylate: 3,4-dimethacryloyloxytetrahydrofuran

### Referential Example 1

Synthesis of Erythritan (cis-3,4-dihydroxytetrahydrofuran)>

Into a reaction vessel fitted with a distillation-cooling part were charged 30.0 g (246 mmol) of erythritol and 0.50 g (2.6 mmol, 1.1 molar equivalents) of p-toluenesulfonic acid monohydrate. Then, the pressure in the system was reduced to 70 Pa and the whole was heated on an oil bath to initiate the reaction. When a period of 25 minutes was passed, a cyclic product erythritan started to distill and a fraction having a distillation temperature of 105 to 107° C. was obtained by fractionation (18.22 g; yield 71.1%). When it was analyzed by gas chromatography, an area purity was 99.4% and it was found that it contained 16 mol % of H₂O on ¹H-NMR.

A 5.03 g portion of thus obtained hydrated erythritan was diluted with 20 g of toluene and then the toluene was removed by distillation by means of a rotary evaporator. This operation was repeated further twice. Drying under reduced pressure at 40° C. afforded 5.00 g of dehydrated erythritan. When measured on ¹H-NMR, it was found that the water content thereof was less than 1 mol %.

### Example 1

### <Production of Erythritan Monomethacrylate, Erythritan Dimethacrylate; Methacryloyl Chloride Process, Use of Hydrated Erythritan>

Into a reactor thorough which nitrogen was passed were charged 1.00 g (9.61 mmol) of erythritan containing 10 mol % of water, 1.46 g (14.4 mmol, 1.5 eq) of triethylamine, and 5 g of methyl isobutyl ketone (MIBK), followed by cooling with salt-ice so that the temperature in the system became −10° C. Thereto was slowly added dropwise 0.835 g (7.99 mmol, 0.83 eq. to erythritan) of methacryloyl chloride (purity 82%) over a period of 15 minutes. After the dropwise addition, the reaction was continued for 10 hours with slowly elevating the temperature to 0° C. After completion of the reaction, the reaction solution was poured into 10 mL of water and extracted with 20 mL of ethyl acetate. The ethyl acetate layer was washed with 5 mL of 1N HCl twice, 5 mL of a saturated sodium hydrogen carbonate aqueous solution once, and 5 mL of a saturated sodium chloride aqueous solution once and then dried over magnesium sulfate. After drying, ethyl acetate was removed by distillation by means of a rotary evaporator to obtain 709 mg of a light yellow oil.

It was dissolved in a mixed solvent of 9 mL of water-3 mL of methanol, followed by extraction with 9 mL of heptane four times.

Sodium chloride was added to the water-methanol layer in an amount more than its soluble amount and the layer was extracted 3 times with 20 mL of ethyl acetate, followed by drying over magnesium sulfate. After drying, ethyl acetate was removed by distillation by means of a rotary evaporator to obtain 377 mg of a colorless oil. When it was analyzed on ¹H-NMR, ¹³C-NMR, and GC-Mass spectrometry, it was revealed that it was monomethacrylate of erythritan (yield based on methacryloyl chloride 27.4%, GC purity: 100%, GPC purity: 80%).

The heptane solutions used for extraction were combined and then dried over magnesium sulfate. After drying, heptane was removed by distillation by means of a rotary evaporator to obtain 246 mg of a light yellow oil. When it was analyzed on ¹H-NMR, ¹³C-NMR, and GC-Mass spectrometry, it was revealed that it was dimethacrylate of erythritan (yield based on methacryloyl chloride 25.7%, GC purity: 94%, GPC purity: 75%).

### <Erythritan Monomethacrylate>

(¹H-NMR (400 MHz), CDCl₃, in ppm): 6.19 (1H, br S), 5.66 (1H, br S), 5.21 (1H, br dd), 4.50 (1H, br ddd), 4.12 (1H, dd), 4.00 (1H, dd), 3.88 (1H, dd), 3.75 (1H, dd), 2.12 (1H, brs, OH), 1.98 (3H, s, Me)

(¹³C-NMR (100 MHz), CDCl₃, in ppm): 166.96, 135.59, 126.72, 73.97, 72.39, 71.08, 70.56, 18.26

GC-mass (measured after TMS treatment, CI ionization method): M+TMS=245

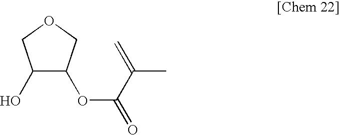

### <Erythritan Dimethacrylate>

(¹H-NMR (400 MHz), CDCl₃, in ppm): 6.11 (2H, br S), 5.60 (2H, m), 5.40 (2H, br ddd), 4.15 (2H, ddd), 3.88 (2H, dd), 1.92 (6H, s)

(¹³C-NMR (100 MHz), CDCl₃, in ppm): 166.68, 135.97, 126.71, 72.13, 70.76, 18.45

GC-mass (measured after TMS treatment, CI ionization method): M+H=241

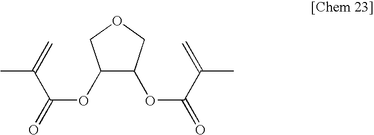

### Example 2

### <Production of Erythritan Monomethacrylate, Erythritan Dimethacrylate; Methacryloyl Chloride Process, Use of Lowly Hydrated Erythritan>

The reaction and purification were carried out in the same manner except than water contained in the erythritan was less than 1 mol % and the amount of the methacryloyl chloride used was 0.895 g (8.56 mmol, 0.89 eq. to erythritan) to obtain the following products.
Erythritan monomethacrylate of a colorless oil 501 mg (yield based on methacryloyl chloride 34%, GC purity: 96%, GPC purity: 81%).
Erythritan dimethacrylate of a light yellow oil 412 mg (yield based on methacryloyl chloride 40.1%, GC purity: 89%, GPC purity: 98%).

### Example 3

### Production of Erythritan Monomethacrylate; Methacryloyl Chloride Process, Use of Low Purity Methacryloyl Chloride>

Into a reactor thorough which nitrogen was passed were charged 15.34 g (144 mmol) of erythritan (water content: less than 1 mol %), 8.01 g (79.2 mmol, 0.55 eq) of triethylamine, and 100 ml of THF, followed by cooling with salt-ice so that the temperature in the system became −5° C. Thereto was slowly added dropwise 7.95 g (73.7 mmol, 0.51 eq. to erythritan) of methacryloyl chloride (purity 85%) over a period of 30 minutes. After the dropwise addition, the reaction was continued for 1 hour with slowly elevating the temperature to 20° C. After completion of the reaction, the reaction solution was poured into 30 mL of a saturated sodium hydrogen carbonate aqueous solution and THF was removed by distillation by means of rotary evaporator. The remaining aqueous layer was extracted with 50 mL of ethyl acetate three times. The ethyl acetate layer was washed with 30 mL of 1N HCl aqueous solution once, 30 mL of a saturated sodium hydrogen carbonate aqueous solution three times, and 30 mL of a saturated sodium chloride aqueous solution three times and then dried over magnesium sulfate. After drying, ethyl acetate was removed by distillation by means of rotary evaporator to obtain 6.67 g of a yellow oil.

It was dissolved in a mixed solvent of 60 mL of water-30 mL of methanol, followed by extraction with 30 mL of heptane three times. After methanol was removed by distillation from the water-methanol layer by means of a rotary evaporator, the layer was extracted with 30 mL of ethyl acetate three times, followed by drying over magnesium sulfate. After drying, ethyl acetate was removed by distillation by means of a rotary evaporator to obtain 4.00 g of a yellow oil (yield based on methacryloyl chloride 31.5%). It was distilled on a thin-film distillation apparatus where temperature of a vaporizing part was set at 98° C. Erythritan monomethacrylate was obtained in an amount of 2.85 g from the distilled part as a colorless oil (yield based on methacryloyl chloride 22.5%, GC purity: 94%, GPC purity: 87%). Another component in GPC was a compound of the formula (4) (wherein R⁴═H, R⁵=(6) or (7)) and the GPC area ratio thereof was 13%.

### Example 4

### <Production of Erythritan Monomethacrylate; Methacryloyl Chloride Process, Use of High Purity Methacryloyl Chloride>

Into a reactor thorough which nitrogen was passed were charged 20.00 g (192 mmol) of erythritan (water content: less than 1 mol %), 15.50 g (153 mmol, 0.797 eq) of triethylamine, and 150 ml of THF, followed by cooling with salt-ice so that the temperature in the system became −5° C. Thereto was slowly added dropwise 13.79 g (128 mmol, 0.666 eq. to erythritan) of methacryloyl chloride (purity 97%) over a period of 60 minutes. After the dropwise addition, the reaction was continued for 1 hour with slowly elevating the temperature to 20° C. After completion of the reaction, the reaction solution was poured into 20 mL of a saturated sodium hydrogen carbonate aqueous solution and then THF was removed by distillation by means of rotary evaporator. The remaining aqueous layer was extracted with 50 mL of ethyl acetate three times. The ethyl acetate layer was washed with 20 mL of 1N HCl aqueous solution once, 20 mL of a saturated sodium hydrogen carbonate aqueous solution twice, and 20 mL of a saturated sodium chloride aqueous solution twice and then dried over magnesium sulfate. After drying, ethyl acetate was removed by distillation by means of rotary evaporator to obtain 15.75 g of a yellow oil.

It was dissolved in a mixed solvent of 140 mL of water-70 mL of methanol, followed by extraction with 70 mL of heptane five times. The water-methanol layer was extracted with 50 mL of ethyl acetate three times, followed by drying over magnesium sulfate. After drying, ethyl acetate was removed by distillation by means of a rotary evaporator to obtain 9.50 g of a colorless oil (yield based on methacryloyl chloride 43.1%). It was distilled on a thin-film distillation apparatus where temperature of a vaporizing part was set at 98° C.

After the thin-film distillation, erythritan monomethacrylate was obtained in an amount of 7.46 g from the distilled part as a colorless oil (yield based on methacryloyl chloride 33.9%, GC purity: 99%, GPC purity: 99%). Another component in GPC was a compound of the formula (4) (wherein R⁴═H, R⁵=(6) or (7)) and the GPC area ratio thereof was 1%.

### Example 5

### <Production of Erythritan Monomethacrylate, Erythritan Dimethacrylate; Methyl Methacrylate Process>

Into a reactor thorough which nitrogen was passed were charged 1.57 g (15.1 mmol) of erythritan (water content: less than 1 mol %), 5.75 g (75.6 mmol, 5 eq) of methyl methacrylate, 5 ml of ethylene glycol dimethyl ether, 2.8 mg of methoxyphenol as a polymerization inhibitor, and 81 mg (1.5 mmol, 0.1 eq.) of sodium methoxide as a catalyst, followed by heating so that the temperature in the system became 85° C. The reaction was continued for 18 hours with removing an azeotropic mixture of distilled methanol and methyl methacrylate from the system by distillation. After completion of the reaction, the reaction solution was poured into 10 mL of 1N hydrochloric acid, followed by extraction with 10 mL of ethyl acetate three times. The ethyl acetate layer was washed with 20 mL of a saturated sodium hydrogen carbonate aqueous solution once and 10 mL of a saturated sodium chloride aqueous solution once and then dried over magnesium sulfate. After drying, ethyl acetate was removed by distillation by means of rotary evaporator to obtain 1.30 g of a light yellow oil. When it was measured on ¹H-NMR, the ratio of erythritan monomethacrylate/erythritan dimethacrylate was 78/22 and respective yields calculated backward based thereon were as follows: erythritan monomethacrylate: 36%, erythritan dimethacrylate 10%. Moreover, as a result of GC analysis, purity of erythritan monomethacrylate and erythritan dimethacrylate in total was 94%.

### Example 6

### <Production of Erythritan Monomethacrylate; Methacryloyl Chloride-K2CO3 Process, Use of High Purity Methacryloyl Chloride>

Into a reactor thorough which nitrogen was passed were charged 75.12 g (722 mmol) of erythritan (water content: less than 1 mol %), 110.00 g (796 mmol, 1.10 eq) of anhydrous potassium carbonate, and 560 ml of acetonitrile, followed by cooling with salt-ice so that the temperature in the system became 0° C. Thereto was slowly added dropwise 75.00 g (720 mmol, 1.00 eq. to erythritan) of methacryloyl chloride (purity 99%) over a period of 90 minutes. After the dropwise addition, the reaction was continued for 30 minutes as it was and then further for 2 hours with slowly elevating the temperature to 20° C. After completion of the reaction, the reaction solution was filtrated to remove salts and then 150 mL of a 3 wt % sodium hydrogen carbonate aqueous solution was added thereto, followed by removal of acetonitrile by distillation by means of rotary evaporator. The remaining aqueous layer was extracted with 225 mL of ethyl acetate twice. After the ethyl acetate layer was washed with 75 mL of a saturated sodium chloride aqueous solution twice, ethyl acetate was removed by distillation by means of rotary evaporator to obtain 126 g of a colorless transparent oil.

It was dissolved in a mixed solvent of 75 mL of water-38 mL of methanol, followed by extraction with 150 mL of heptane seven times. Methanol was removed by distillation from the water-methanol layer by means of a rotary evaporator to obtain 120 g of a colorless transparent oil.

Then, the water-methanol layer was extracted with 150 mL of ethyl acetate twice and 8 mg of 2,2,6,6-tetramethylpiperidine-1-oxyl free radical (TEMPO) was added as a polymerization inhibitor. Ethyl acetate was removed by distillation by means of rotary evaporator to obtain 85.69 g of a colorless oil (yield based on methacryloyl chloride 69.1%). It was distilled on a thin-film distillation apparatus where temperature of a vaporizing part was set at 98° C. After the thin-film distillation, erythritan monomethacrylate was obtained in an amount of 77.62 g from the distilled part as a colorless oil (yield based on methacryloyl chloride 62.6%, GC purity: 98.5%, GPC purity: >99%).

### Example 7

### <Production of Erythritan Monomethacrylate; Methacrylic Anhydride Process>

Into a reactor thorough which nitrogen was passed were charged 18.55 g (182 mmol) of erythritan (water content: less than 1 mol %), 12.24 g (121 mmol, 0.664 eq) of triethylamine, 1.48 g (12.1 mmol, 0.066 eq.) of dimethylaminopyridine, and 138 ml of acetonitrile, followed by cooling with salt-ice so that the temperature in the system became −5° C. Thereto was slowly added dropwise 18.65 g (121 mmol, 0.664 eq. to erythritan) of methacrylic anhydride over a period of 40 minutes. After the dropwise addition, the temperature was slowly elevated to 20° C., followed by standing overnight as it was. After completion of the reaction, 40 mL of a saturated sodium hydrogen carbonate aqueous solution was added to the reaction solution, followed by removal of acetonitrile by distillation by means of rotary evaporator. The remaining aqueous layer was extracted with 50 mL of ethyl acetate three times. The ethyl acetate layer was washed with 20 mL of 1N HCl aqueous solution twice, 20 mL of a saturated sodium hydrogen carbonate aqueous solution twice, and 20 mL of a saturated sodium chloride aqueous solution twice. Then, ethyl acetate was removed by distillation by means of rotary evaporator to obtain 15.75 g of a pale yellow oil.

It was dissolved in a mixed solvent of 40 mL of water-20 mL of methanol, followed by extraction with 40 mL of heptane five times. Methanol was removed by distillation from the water-methanol layer by means of a rotary evaporator to obtain 42.35 g of a colorless transparent oil.

Then, the aqueous layer was extracted with 40 mL of ethyl acetate three times, followed by drying over magnesium sulfate. After drying, 5 mg of 2,2,6,6-tetramethylpiperidine-1-oxyl free radical (TEMPO) was added as a polymerization inhibitor. Ethyl acetate was removed by distillation by means of rotary evaporator to obtain 11.60 g of a colorless oil (yield based on methacrylic anhydride 54.6%). It was distilled on a thin-film distillation apparatus where temperature of a vaporizing part was set at 98° C.

After the thin-film distillation, erythritan monomethacrylate was obtained in an amount of 9.70 g from the distilled part as a colorless oil (yield based on methacrylic anhydride 46.6%, GC purity: 97.7%, GPC purity: >99%).

### Example 8

### <Production of Erythritan Monomethacrylate; DCC Process>

Into a reactor thorough which nitrogen was passed were charged 18.73 g (185 mmol) of erythritan (water content: less than 1 mol %), 10.30 g (121 mmol, 0.654 eq) of methacrylic acid, 1.18 g (9.68 mmol, 0.052 eq) of dimethylaminopyridine, 10 mg of phenothiazine (1000 ppm relative to methacrylic acid), and 150 ml of methylene chloride, followed by cooling with ice so that the temperature in the system became 3° C. Thereto was slowly added dropwise a solution of 25.00 g (121 mmol, 0.654 eq.) of N,N′-dicyclohexylcarbodiimide (DCC) in 10 mL of methylene chloride over a period of 30 minutes. After the dropwise addition, the reaction was continued for 30 minutes as it was and then temperature was slowly elevated to 20° C., followed by standing as it was for 2 days. After completion of the reaction, precipitated urea was filtrated and the reaction solution was washed with 50 mL of a 1N HCl aqueous solution once, 50 mL of a saturated sodium hydrogen carbonate aqueous solution twice, and 50 mL of a saturated sodium chloride aqueous solution twice. Then, methylene chloride was removed by distillation by means of rotary evaporator to obtain 20.00 g of a pale orange oil.

It was dissolved in a mixed solvent of 50 mL of water-100 mL of methanol, followed by extraction with 50 mL of heptane three times. Methanol was removed by distillation from the water-methanol layer by means of a rotary evaporator to obtain a colorless transparent oil.

Then, the aqueous layer was extracted with 50 mL of ethyl acetate three times, followed by drying over magnesium sulfate. After drying, 5 mg of tetramethylpiperidine N-oxyl was added as a polymerization inhibitor. Ethyl acetate was removed by distillation by means of rotary evaporator to obtain 13.52 g of a pale orange oil (yield based on methacrylic anhydride 65.0%). It was distilled on a thin-film distillation apparatus where temperature of a vaporizing part was set at 98° C.

After the thin-film distillation, erythritan monomethacrylate was obtained in an amount of 10.30 g from the distilled part as a colorless oil (yield based on methacrylic anhydride 55.6%, GC purity: 97.5%).

### Example 9

### <Solubility Test of Erythritan Monomethacrylate (HOTHFMA)>

A solubility test of erythritan monomethacrylate (HOTHFMA) in aqueous solvents shown in the following table was carried out at a temperature of 26° C. In this connection, for the purpose of comparison, data on tetrahydrofurfuryl methacrylate (TFMA) and α-methacryloyl-γ-butyrolactone (GBLMA) are also shown. The numeric values in the table represent grams of individual solutes dissolved in 100 g of each solvent.

It is realized that erythritan monomethacrylate of the invention has a remarkably high solubility to aqueous solvents as compared with tetrahydrofurfuryl methacrylate having the same tetrahydrofuran skeleton. Moreover, it is realized that erythritan monomethacrylate shows an excellent solubility to both of heptane and H₂O/MeOH (4/1) in comparison with GBLMA.

TABLE 1
Solubility (26° C.): g/100 g solv.

| | | H2O/MeOH | |
|---|---|---|---|
| | Heptane | 4/1 (wt) | H2O |
| GBLMA | 1.00 | 3.22 | — |
| HOTHFMA | 1.74 | >353 | >395 |
| TFMA | >50 | 5.11 | 2.55 |

### Referential Example 2

<Synthesis of 3-methacryloyloxy-4-hydroxytetrahydrofuran>

Into a reactor thorough which nitrogen was passed were charged 30.0 g (288 mmol) of 3,4-dihydroxytetrahydrofuran (erythritan), 44 g of potassium carbonate (K₂CO₃; 317 mmol, 1.1 eq), 225 mL of acetonitrile, and 30 mg of 4-methoxyphenol as a polymerization inhibitor, followed by cooling on an ice bath under stirring so that the temperature in the system became 5° C. Thereto was added dropwise a solution of 30.0 g (288 mmol, 1.0 eq. to erythritan) of methacryloyl chloride dissolved in 6 mL of acetonitrile so that the temperature in the system was maintained at 5° C. or lower. After the dropwise addition, the whole was stirred at 5° C. for 30 minutes and then the temperature was elevated to 20° C., followed by stirring for another 2 hours. After completion of the reaction, 6 mL of a 5% NaHCO₃ aqueous solution was added to the reaction solution to quench a minute amount of remaining methacryloyl chloride and then an inorganic salt formed during the reaction was filtrated. After 54 mL of a 5% NaHCO₃ aqueous solution was added to the resulting filtrate, it was concentrated by means of an evaporator to obtain 99.8 g of a concentrate. When analyzed, it contained 6.2% by weight of acetonitrile and 50.9% by weight of water. Moreover, when the solution was analyzed by gas chromatography (FID detector), the area ratio of 3-methacryloyloxy-4-hydroxytetrahydrofuran to 3,4-dimethacryloyloxytetrahydrofuran was 96.7/3.3.

The concentrate was divided into samples of 16.3 g each, which were used in the following Examples 10 to 12.

### Example 10

<Removal of 3,4-dimethacryloyloxytetrahydrofuran by Extraction>

To 16.3 g of the sample of Referential Example 2 were added 3.3 g of acetonitrile and 10 g of water (content of acetonitrile relative to water was 23.7% by weight). The solution was extracted with 10 mL of heptane three times to remove 3,4-dimethacryloyloxytetrahydrofuran. Furthermore, the solution was extracted with 20 mL of AcOEt three times and the resulting AcOEt layer was washed with 8 mL of water twice and the solvent was removed to obtain 5.2 g of a colorless oil (isolation yield from the reaction: 63%). When the oil was analyzed by gas chromatography (FID detector), the area ratio of 3-methacryloyloxy-4-hydroxytetrahydrofuran to 3,4-dimethacryloyloxytetrahydrofuran was 98.9/1.1.

### Example 11

<Removal of 3,4-dimethacryloyloxytetrahydrofuran by Extraction>

The same operations as in Example 10 were performed except that 10 g of water alone was added to 16.6 g of the sample of Referential Example 2 (content of acetonitrile relative to water was 5.5% by weight) to obtain 4.9 g of a colorless oil (isolation yield from the reaction: 59%). When the oil was analyzed by gas chromatography (FID detector), the area ratio of 3-methacryloyloxy-4-hydroxytetrahydrofuran to 3,4-dimethacryloyloxytetrahydrofuran was 99.5/0.5.

### Example 12

<Removal of 3,4-dimethacryloyloxytetrahydrofuran by Extraction>

The same operations as in Example 10 were performed except that 20 g of water alone was added to 16.6 g of the sample of Referential Example 2 (content of acetonitrile relative to water was 3.6% by weight) to obtain 4.8 g of a colorless oil (isolation yield from the reaction: 58%). When the oil was analyzed by gas chromatography (FID detector), the area ratio of 3-methacryloyloxy-4-hydroxytetrahydrofuran to 3,4-dimethacryloyloxytetrahydrofuran was 99.6/0.4.

### Example 13

<Removal of 3,4-dimethacryloyloxytetrahydrofuran by Extraction>

The same operations as in Example 10 were performed except that 6.6 g of acetonitrile and 10 g of water were added to 16.6 g of the sample of Referential Example 2 (content of acetonitrile relative to water was 42.2% by weight) to obtain 4.9 g of a colorless oil (isolation yield from the reaction: 59%). When the oil was analyzed by gas chromatography (FID detector), the area ratio of 3-methacryloyloxy-4-hydroxytetrahydrofuran to 3,4-dimethacryloyloxytetrahydrofuran was 96.8/3.2.

From the above results, it is found that an objective compound containing substantially reduced di(meth)acryloyloxytetrahydrofuran which is a serious changing factor of polymerization degree is obtained when a solution having a content of an aprotic polar solvent miscible with water of 40% by weight or less relative to the amount of water present in the system is subjected to an extraction treatment with a solvent containing a hydrocarbon.

### Referential Example 3

<Synthesis of 3-hydroxy-4-methacryloyloxytetrahydrofuran>

Into a reactor thorough which nitrogen was passed were charged 104.1 g (1.0 mol) of 3,4-dihydroxytetrahydrofuran, 152.0 g of potassium carbonate (K₂CO₃; 1.1 mol, 1.1 eq), and 780 mL of acetonitrile, followed by cooling under stirring so that the temperature in the system became 0° C. Thereto was added dropwise 104.5 g (1.0 mol, 1.0 eq. to erythritan, containing a stabilizer: phenothiazine=1000 ppm) of methacryloyl chloride so that the temperature in the system was maintained at 5° C. or lower. After the dropwise addition, the whole was stirred at 5° C. for 2 hours. After completion of the reaction, 20 mL of a 5% NaHCO₃ aqueous solution was added to the reaction solution to quench a minute amount of remaining methacryloyl chloride and then an inorganic salt formed during the reaction was filtrated. After 190 mL of a 5% NaHCO₃ aqueous solution and 5 mg of tetramethylpiperidinyl-N-oxide were added to the resulting filtrate, the resultant was concentrated by means of an evaporator to obtain 268 g of a concentrate. When analyzed, it contained 5% by weight of acetonitrile and 50% by weight of water. Moreover, when the solution was analyzed by gas chromatography (FID detector), the area ratio of 3-methacryloyloxy-4-hydroxytetrahydrofuran to 3,4-dimethacryloyloxytetrahydrofuran was 94.5/5.5.

### Example 14

<Removal of 3,4-dimethacryloyloxytetrahydrofuran by Extraction>

To 69.9 g of the sample of Referential Example 3 was added 52 g of water (content of acetonitrile relative to water was 6.7% by weight). Then, 52 mL of heptane and 20 mL of ethyl acetate were added to the solution and an upper layer (heptane-ethyl acetate layer) containing 3,4-dimethacryloyloxytetrahydrofuran was removed by extraction. Thereafter, a lower layer (aqueous layer) was extracted with 80 mL of AcOEt three times, the resulting ethyl acetate layer was washed with 26 mL of water twice, and the solvent was removed to obtain 23.3 g of a colorless oil (isolation yield from the reaction: 52%). When the oil was analyzed by gas chromatography (FID detector), the area ratio of 3-methacryloyloxy-4-hydroxytetrahydrofuran to 3,4-dimethacryloyloxytetrahydrofuran was 99.0/1.0.

### Example 15

<Removal of 3,4-dimethacryloyloxytetrahydrofuran by Extraction>

To 69.9 g of the sample of Referential Example 3 was added 52 g of water (content of acetonitrile relative to water was 6.7% by weight). Then, 52 mL of heptane and 20 mL of ethyl acetate were added to the solution, extraction was performed, and an upper layer (heptane-ethyl acetate layer) was removed. Again, 52 mL of heptane and 20 mL of ethyl acetate were added to the resulting lower layer (aqueous layer), extraction was performed, and an upper layer (heptane-ethyl acetate layer) was removed. Thereafter, the same operations as in Example 13 were performed to obtain 18.9 g of a colorless oil (isolation yield from the reaction: 42%). When the oil was analyzed by gas chromatography (FID detector), the area ratio of 3-methacryloyloxy-4-hydroxytetrahydrofuran to 3,4-dimethacryloyloxytetrahydrofuran was 99.7/0.3.

While the invention has been described in detail and with reference to specific embodiments thereof, it will be apparent to one skilled in the art that various changes and modifications can be made therein without departing from the spirit and scope thereof.

The present application is based on Japanese Patent Application No. 2004-224315 filed on Jul. 30, 2004, Japanese Patent Application No. 2005-181206 filed on Jun. 21, 2005, Japanese Patent Application No. 2005-216420 filed on Jul. 26, 2005, Japanese Patent Application No. 2005-307683 filed on Oct. 21, 2005, and Japanese Patent Application No. 2005-147710 filed on May 20, 2005, and the contents are incorporated herein by reference.

### INDUSTRIAL APPLICABILITY

The (meth)acryloyloxytetrahydrofuran of the invention has a structure containing a heterocyclic ring and a hydrophilic group in the same monomer molecule and actually exhibits an extremely good solubility to aqueous solvents and thus a high hydrophilicity to water, so that it is possible to utilize it for the purpose of modification to impart hydrophilicity to various (meth)acrylic resins. Examples of usable fields include resins for resists such as color resists and semiconductor resists, resins for medical materials such as dental materials, resins for paints and coatings, resins for adhesives, and resins for textile treatment. Furthermore, in the recent resist field, a technical development by an immersion method has been actively performed in an image-forming technology by ArF laser and necessity of a topcoat resin capable of being dissolved in an alkaline developing fluid has been increased. Thus, in view of the characteristics of the compound of the invention, it might be also possible to apply it to this application.

Additionally, according to the production process of the invention, it is possible to produce the polymerizable monomer rich in hydrophilicity in good yields by industrially simple and convenient operations.

1. A (meth)acryloyloxytetrahydrofuran having a structure represented by general formula (1): 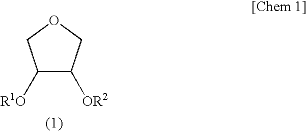
wherein R¹ and R² each is a hydrogen atom or a (meth)acryloyl group represented by general formula (2) and at least one of R¹ and R² is a (meth)acryloyl group represented by general formula (2): 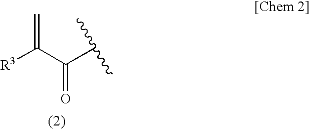
wherein R³ is a hydrogen atom or a methyl group and the wavy line represents a bonding site.

2. A process for producing the (meth)acryloyloxytetrahydrofuran according to claim 1, which comprises reacting 3,4-dihydroxytetrahydrofuran represented by formula (3) with a (meth)acryloyl halide in the presence of a basic substance:

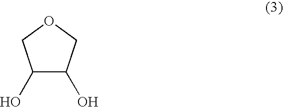

3. The process for producing the (meth)acryloyloxytetrahydrofuran according to claim 2, wherein the reaction is carried out further in the presence of an aprotic polar solvent miscible with water.

4. The process for producing the (meth)acryloyloxytetrahydrofuran according to claim 3, wherein the aprotic polar solvent miscible with water is at least one solvent selected from ketones, ethers, nitrites, amides, and sulfoxides, each of which has 10 or less carbon atoms.

5. The process for producing the (meth)acryloyloxytetrahydrofuran according to claim 2, wherein a (meth)acryloyl halide having a purity of 85% by mol or more is used as the (meth)acryloyl halide of a raw material.

6. The process for producing the (meth)acryloyloxytetrahydrofuran according to claim 3, wherein a (meth)acryloyl halide containing a dimer of (meth)acryloyl halide in an amount of 15% by mol or less is used as the (meth)acryloyl halide of a raw material.

7. The process for producing the (meth)acryloyloxytetrahydrofuran according to claim 2, wherein the amount of water contained in 3,4-dihydroxytetrahydrofuran of a raw material is 10% by mol or less relative to 3,4-dihydroxytetrahydrofuran.

8. A (meth)acryloyloxytetrahydrofuran composition, wherein each content of the compounds having structures represented by general formulae (4) and (5) in the general formula (1) is 10% or less as an area ratio when analyzed by gel permeation chromatography with an RI detector: 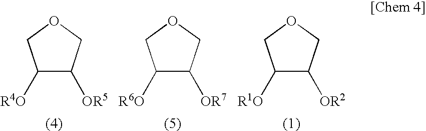
wherein R¹ and R² each is a hydrogen atom or a (meth)acryloyl group represented by formula (2) and at least one of R¹ and R² is a (meth)acryloyl group represented by general formula (2): 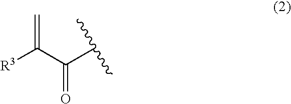
wherein R³ is a hydrogen atom or a methyl group and the wavy line represents a bonding site, R⁴ is a hydrogen atom or a (meth)acryloyl group represented by general formula (2) and R⁵, R⁶, and R⁷ each represents a group represented by general formula (6) or (7): 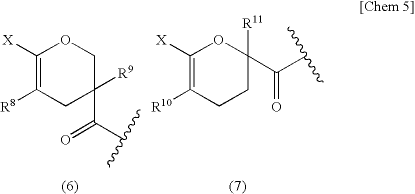
wherein R⁸, R⁹, R¹⁰ and R¹¹ each is a hydrogen atom or a methyl group, X represents a halogen atom, and the wavy line represents a bonding site.

9. A process for producing a (meth)acryloyloxytetrahydrofuran, which comprises removing a di(meth)acryloyloxytetrahydrofuran by extraction with an extraction solvent containing water and a hydrocarbon solvent from a (meth)acryloyloxytetrahydrofuran composition containing a (meth)acryloyloxytetrahydrofuran having a structure represented by general formula (8) and a di(meth)acryloyloxytetrahydrofuran represented by general formula (9): 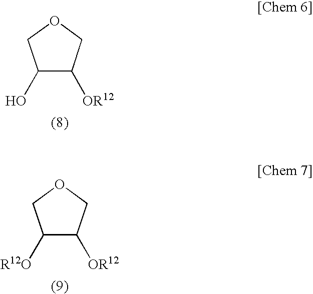
wherein R¹² is a (meth)acryloyl group represented by general formula (2): 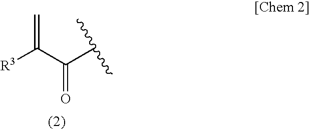
wherein R³ is a hydrogen atom or a methyl group and the wavy line represents a bonding site.

10. The process for producing a (meth) acryloyloxytetrahydrofuran according to claim 9, wherein an extraction solvent containing an aprotic polar solvent miscible with water is further used.

11. The process for producing a (meth)acryloyloxytetrahydrofuran according to claim 9, wherein the (meth)acryloyloxytetrahydrofuran composition is obtained by reacting 3,4-dihydroxytetrahydrofuran represented by formula (3): 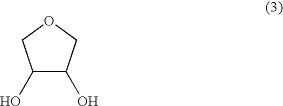
with a (meth)acryloyl halide in the presence of a basic substance.

12. The process for producing a (meth)acryloyloxytetrahydrofuran according to claim 11, wherein the reaction is carried out further in the presence of an aprotic polar solvent miscible with water.

13. The process for producing a (meth)acryloyloxytetrahydrofuran according to claim 10, wherein the content of the aprotic polar solvent miscible with water is 40% by weight or less relative to the weight of water.

14. The process for producing a (meth)acryloyloxytetrahydrofuran according to claim 10, wherein the aprotic polar solvent miscible with water is at least one solvent selected from ketones, ethers, nitrites, amides, and sulfoxides, each of which has 10 or less carbon atoms.

15. A 3-(meth)acryloyloxy-4-hydroxytetrahydrofuran composition, wherein the abundance ratio of a (meth)acryloyloxytetrahydrofuran represented by general formula (8) to a di(meth)acryloyloxytetrahydrofuran represented by general formula (9): 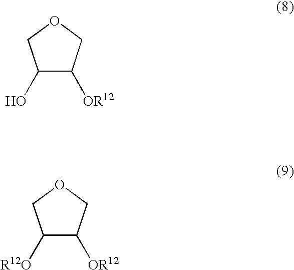
is 97/3 or more as an area ratio when analyzed by gas chromatography with an FID detector wherein R¹² is a (meth)acryloyl group represented by general formula (2) 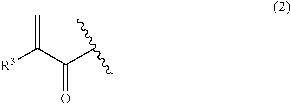
wherein R³ is a hydrogen atom or a methyl group and the wavy line represents a bonding site.

16. A resist resin composition comprising a (meth)acryloyloxytetrahydrofuran represented by general formula (1) as a constitutional component: 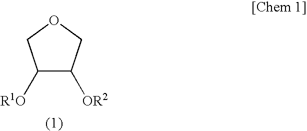
wherein R¹ and R² each is a hydrogen atom or a (meth)acryloyl group represented by general formula (2) and at least one of R¹ and R² is a (meth)acryloyl group represented by general formula (2): 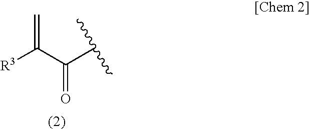
wherein R³ is a hydrogen atom or a methyl group and the wavy line represents a bonding site.

17. The resist resin composition according to claim 16, which comprises, as a constitutional component, an acid-dissociating monomer as a copolymer composition.
